(19) United States
(12) Patent Application Publication
(10) Pub. No.: US 2004/0115707 A1
(43) Pub. Date: Jun. 17, 2004

(54) Biochemical analysis units

(75) Inventor: Yoshikazu Amano, Minamiashigara-shi (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 10/662,825

(22) Filed: Sep. 16, 2003

(30) Foreign Application Priority Data
Sep. 17, 2002 (JP) 269578/2002

(51) Int. Cl. C12Q 1/68; C12M 1/34

(52) U.S. Cl. 435/6; 435/287.2

(57) ABSTRACT

A biochemical analysis unit comprises a base plate, which has a plurality of holes and is constituted of a material having radiation attenuating properties and/or light attenuating properties, and a porous adsorptive material, which is filled in each of the plurality of the holes of the base plate and forms each of a plurality of adsorptive regions. The porous adsorptive material, which forms each of the plurality of the adsorptive regions, has a pore diameter falling within the range of 1 μm to 10 μm. The porous adsorptive material may take on the form of a film.

### BACKGROUND OF THE INVENTION

1. Field of the Invention

This invention relates to a biochemical analysis unit for use in an operation for detecting a labeled receptor or a labeled ligand.

2. Description of the Related Art

As techniques for analyzing gene abnormality related to various diseases, various techniques, such as a Northern blotting technique, a dot blotting hybridization technique, and a Southern blotting technique, have heretofore been known widely. With the Northern blotting technique, an mRNA fraction is extracted from a tissue and subjected to agarose gel electrophoresis, and the mRNA having migrated is transferred to a nitrocellulose film, or the like, with a blotting process. Thereafter, the mRNA is fixed to the film, and a complementary DNA probe, which has been labeled with radiation, is subjected to hybridization with the mRNA. Also, X-ray film is exposed to the radiation coming from the probe, and autoradiography is thus performed. With the dot blotting hybridization technique, instead of an mRNA fraction being subjected to electrophoresis, the mRNA fraction is directly spotted onto a nitrocellulose film, or the like, a DNA probe is subjected to hybridization with the mRNA, and the quantity of the mRNA is determined. With the Southern blotting technique, a chromosome DNA is extracted from a tissue, broken with a restriction enzyme, and subjected to agarose gel electrophoresis. Also, the DNA fragment having migrated is transferred to a nitrocellulose film, or the like. Thereafter, the DNA fragment is fixed to the film, and a complementary DNA probe, which has been labeled with radiation, is subjected to hybridization with the DNA fragment. Also, X-ray film is exposed to the radiation coming from the probe, and autoradiography is thus performed.

Also, various biochemical analysis systems have heretofore been used. With the biochemical analysis systems, liquids containing ligands or receptors (i.e., the substances, which are capable of specifically binding to organism-originating substances and whose base sequences, base lengths, compositions, characteristics, and the like, are known) are spotted onto different positions on a surface of a supporting material of a glass array, which utilizes a slide glass plate, or the like, or a membrane array, which utilizes a membrane filter, or the like, and a plurality of spot-shaped regions are thereby formed on the surface of the supporting material. Examples of the ligands or the receptors include hormones, tumor markers, enzymes, antibodies, antigens, abzymes, other proteins, nucleic acids, cDNA's, DNA's, and RNA's. Thereafter, a labeled receptor or a labeled ligand, which has been labeled with a radioactive labeling substance, a fluorescent labeling substance, a labeling substance capable of producing chemical luminescence when being brought into contact with a chemical luminescence substrate, or the like, is subjected to hybridization, or the like, with the ligands or the receptors, which are contained in the spot-shaped regions of the supporting material. The labeled receptor or the labeled ligand is thus specifically bound to one of the ligands or the receptors, which are contained in the spot-shaped regions of the supporting material. The labeled receptor or the labeled ligand is the substance, which has been sampled from an organism through extraction, isolation, or the like, or has been subjected to chemical treatment after being sampled, and which has been labeled with the radioactive labeling substance, the fluorescent labeling substance, the labeling substance capable of producing the chemical luminescence when being brought into contact with a chemical luminescence substrate, or the like. Examples of the labeled receptors or the labeled ligands include hormones, tumor markers, enzymes, antibodies, antigens, abzymes, other proteins, nucleic acids, DNA's, and mRNA's.

In cases where the labeled receptor or the labeled ligand has been labeled with the radioactive labeling substance, a stimulable phosphor layer of a stimulable phosphor sheet is then exposed to radiation radiated out from the radioactive labeling substance, which is contained selectively in the spot-shaped region of the supporting material. Thereafter, the stimulable phosphor layer is exposed to stimulating rays, which cause the stimulable phosphor layer to emit light in proportion to the amount of energy stored on the stimulable phosphor layer during the exposure of the stimulable phosphor layer to the radiation. The light emitted by the stimulable phosphor layer is detected photoelectrically.

In cases where the labeled receptor or the labeled ligand has been labeled with the fluorescent labeling substance, excitation light is irradiated to the spot-shaped regions of the supporting material, and the fluorescent labeling substance, which is contained selectively in the spot-shaped region of the supporting material, is excited by the excitation light to produce fluorescence. The thus produced fluorescence is detected photoelectrically.

In cases where the labeled receptor or the labeled ligand has been labeled with the labeling substance capable of producing the chemical luminescence when being brought into contact with a chemical luminescence substrate, the labeling substance, which is contained selectively in the spot-shaped region of the supporting material, is brought into contact with the chemical luminescence substrate. Also, the chemical luminescence produced by the labeling substance is detected photoelectrically.

With the biochemical analysis systems described above, a large number of the ligands or the receptors are formed at a high density at different positions on the surface of the supporting material of the membrane filter, or the like, and the labeled receptor or the labeled ligand, which has been labeled with the labeling substance, such as the fluorescent labeling substance, is subjected to the hybridization, or the like, with the ligands or the receptors, which have been formed at a high density at different positions on the surface of the supporting material. Therefore, the biochemical analysis systems described above have the advantages in that an organism-originating substance is capable of being analyzed quickly.

The biochemical analysis systems described above are required to enable the detection with a sufficiently high accuracy, an enhanced detection limit, and enhanced reproducibility. However, with the biochemical analysis systems, wherein the labeled receptor or the labeled ligand, which has been labeled with the fluorescent labeling substance, is detected by the utilization of the ligands or the receptors, which have been fixed to the glass array, since the detection sensitivity is low, it is necessary that a large amount of the labeled receptor or a large amount of the labeled ligand be utilized for the expression analysis. Also, with the biochemical analysis systems, wherein the labeled receptor or the labeled ligand, which has been labeled with the fluorescent labeling substance, is detected by the utilization of the ligands or the receptors, which have been fixed to the glass array, the problems occur in that, for example, the amount of each of the ligands or the receptors capable of being fixed to the glass array is small, and that the ligands or the receptors having been fixed to the glass array peel off from the glass array during the processes of the analysis operation. With the biochemical analysis systems, wherein the labeled receptor or the labeled ligand, which has been labeled with the labeling substance capable of producing the chemical luminescence when being brought into contact with a chemical luminescence substrate, is detected by the utilization of the ligands or the receptors, which have been fixed to the membrane array, the problems occur in that the detection sensitivity is lower than the detection sensitivity of the biochemical analysis systems utilizing the radioactive labeling substance. With the biochemical analysis systems, wherein the labeled receptor or the labeled ligand, which has been labeled with the radioactive labeling substance, is detected by the utilization of the ligands or the receptors, which have been fixed to the membrane array, a high detection sensitivity is capable of being achieved, but the membrane array is not easy to process.

| [Patent literature 1] | U.S. Patent No 5,543,295 |
|---|---|
| [Non-patent literature 1] | “Nature Genetics,” |
| | Vol. 21, pp. 25-32, |
| | 1999 |
| [Non-patent literature 2] | “Bioindustry,” |
| | Vol. 18, pp. 13-19, |
| | 2001 |

Heretofore, with the biochemical analysis systems described above, the hybridization, or the like, has ordinarily been performed with a shaking technique. With the shaking technique, the experimenter manually puts an array, on which the ligands or the receptors have been fixed, into a hybridization bag and adds a reaction liquid, which contains the labeled receptor or the labeled ligand, into the hybridization bag. Also, the experimenter manually gives vibrations to the hybridization bag, and the labeled receptor or the labeled ligand is thus moved through convection or diffusion. In this manner, the labeled receptor or the labeled ligand is specifically bound to one of the ligands or the receptors having been fixed on the array.

However, with the shaking technique described above, it is not always possible to achieve efficient diffusion of the labeled receptor or the labeled ligand, which is contained in the hybridization reaction liquid, through each of the plurality of the spot-shaped regions, which contain the ligands or the receptors. Therefore, the problems occur in that the ligands or the receptors and the labeled receptor or the labeled ligand cannot efficiently be subjected to the hybridization. In cases where the labeled receptor or the labeled ligand, which is contained in the hybridization reaction liquid, cannot be sufficiently diffused through each of the plurality of the spot-shaped regions, which contain the ligands or the receptors, a ratio of the intensity of the emitted light (signal), which intensity corresponds to the amount of the labeled receptor or the labeled ligand having been bound to the adsorptive region, to the intensity of the emitted light (noise or background) of an adsorptive region, to which the labeled receptor or the labeled ligand has not been bound, cannot be kept high. (The signal-to-noise ratio of the signal representing the intensity of the emitted light, which intensity corresponds to the amount of the labeled receptor or the labeled ligand having been bound to the adsorptive region, to the noise or the background cannot be kept high.) Accordingly, in cases where the amount of the labeled receptor or the labeled ligand, which is bound to the adsorptive region, is small, it becomes difficult for the labeled receptor or the labeled ligand to be detected.

It may be considered that, in order for the labeled receptor or the labeled ligand to penetrate sufficiently into the interior of each of the adsorptive regions of a biochemical analysis unit, the reaction liquid may be forcibly circulated through the interior of each of the adsorptive regions of the biochemical analysis unit. However, ordinarily, the pore diameter of a porous film, which is utilized for a film for fixation of a DNA, or the like, for a gene analysis or a membrane array, is 0.45 μm. Therefore, in cases where the reaction liquid is forcibly circulated through the biochemical analysis unit, which is provided with the adsorptive regions formed on the porous film having a pore diameter described above, and the labeled receptor or the labeled ligand, or the like, is thereby caused to penetrate sufficiently into the interior of each of the adsorptive regions of the biochemical analysis unit, the signal-to-noise ratio cannot be enhanced to an expected level. This is presumably because, with the conventional porous film having the pore diameter described above, since the surface area of each of the adsorptive regions is large, the labeled receptor or the labeled ligand, or the like, is bound to the adsorptive regions in a non-specific manner.

Also, in cases where the reaction liquid is forcibly circulated through the interior of each of the adsorptive regions, it is desired that the flow rate of the reaction liquid is capable of being set as a high rate, and the efficiency of the binding of the labeled receptor or the labeled ligand with the ligands or the receptors is thus capable of being enhanced even further. However, with the conventional porous film having the pore diameter described above, limitation is imposed upon the flow rate of the reaction liquid forcibly circulated through each of the adsorptive regions, and the signal-to-noise ratio cannot be enhanced to an expected level.

### SUMMARY OF THE INVENTION

The primary object of the present invention is to provide a biochemical analysis unit, which enables detection with a high signal-to-noise ratio in cases where a reaction liquid is circulated forcibly through the biochemical analysis unit.

The present invention provides a biochemical analysis unit, comprising:

i) a base plate, which has a plurality of holes and is constituted of a material having radiation attenuating properties and/or light attenuating properties, and

ii) a porous adsorptive material, which is filled in each of the plurality of the holes of the base plate and forms each of a plurality of adsorptive regions,

wherein the porous adsorptive material, which forms each of the plurality of the adsorptive regions, has a pore diameter falling within the range of 1 μm to 10 μm.

In the biochemical analysis unit in accordance with the present invention, the porous adsorptive material, which forms each of the plurality of the adsorptive regions, should preferably have a pore diameter falling within the range of 1 μm to 5 μm, and should more preferably have a pore diameter falling within the range of 2 μm to 4 μm. The term “pore diameter” as used herein means the mean pore diameter of the pores of the porous adsorptive material.

Also, in the biochemical analysis unit in accordance with the present invention, the porous adsorptive material should preferably take on the form of a film.

The conventional porous membrane for biochemical analysis has a pore diameter of as small as 0.45 μm, and therefore the surface area of each of the adsorptive regions of the conventional porous membrane for adsorbing a substance is large. Accordingly, for example, in cases where the conventional porous membrane is utilized for a chemical luminescence technique, the problems occur in that, instead of an enzyme-labeled antibody being bound to an antigen of a labeled receptor or a labeled ligand, the enzyme-labeled antibody is bound directly to the adsorptive regions of the conventional porous membrane in a non-specific manner. As a result, the problems occur in that the intensity of the background becomes high, and therefore the intensity of noise becomes high. However, with the biochemical analysis unit in accordance with the present invention, wherein the porous adsorptive material, which forms each of the plurality of the adsorptive regions, has a pore diameter of at least lam, the problems are capable of being prevented from occurring in that the labeled receptor or the labeled ligand, or the like, is bound to the adsorptive regions in a non-specific manner.

Also, in cases where the pore diameter of the porous adsorptive material is large, the intensity of the background is capable of being kept low. However, if the pore diameter of the porous adsorptive material is very large, the surface area of each of the adsorptive regions for adsorbing a substance will become small, and the amount of each of the ligands or the receptors capable of being fixed to one of the adsorptive regions will become small. Therefore, in such cases, it will be not always possible to detect a small amount of the labeled receptor or the labeled ligand. However, with the biochemical analysis unit in accordance with the present invention, wherein the porous adsorptive material, which forms each of the plurality of the adsorptive regions, has a pore diameter of at most 10 μm, the intensity of the background is capable of being suppressed, and noise is thus capable of being suppressed, while the detection limit is being kept high.

Further, with the biochemical analysis unit in accordance with the present invention, wherein the porous adsorptive material, which forms each of the plurality of the adsorptive regions, has a pore diameter falling within the range of 1 μm to 10 μm, flow characteristics of a reaction liquid, or the like, which flows through each of the adsorptive regions are capable of being enhanced. Therefore, in cases where hybridization, or the like, is performed with a technique, wherein the reaction liquid, or the like, is forcibly circulated through the biochemical analysis unit, such that the reaction liquid, or the like, flows across each of the adsorptive regions of the biochemical analysis unit, and wherein the labeled receptor or the labeled ligand, or the like, is thereby caused to penetrate sufficiently into the interior of each of the adsorptive regions of the biochemical analysis unit, the labeled receptor or the labeled ligand, or the like, which is contained in the reaction liquid, does not clog the adsorptive regions, and the detection of the labeled receptor or the labeled ligand, or the like, is capable of being performed easily.

### BRIEF DESCRIPTION OF THE DRAWINGS

FIG. 1 is a schematic perspective view showing an embodiment of the biochemical analysis unit in accordance with the present invention,

FIGS. 2A and 2B are schematic views showing an example of how the biochemical analysis unit in accordance with the present invention is produced,

FIG. 3 is a schematic view showing a different example of how the biochemical analysis unit in accordance with the present invention is produced, and

FIG. 4 is a schematic sectional view showing an example of a reactor, which is employed for a chemical luminescence method using the biochemical analysis unit in accordance with the present invention.

### DESCRIPTION OF THE PREFERRED EMBODIMENTS

The present invention will hereinbelow be described in further detail with reference to the accompanying drawings.

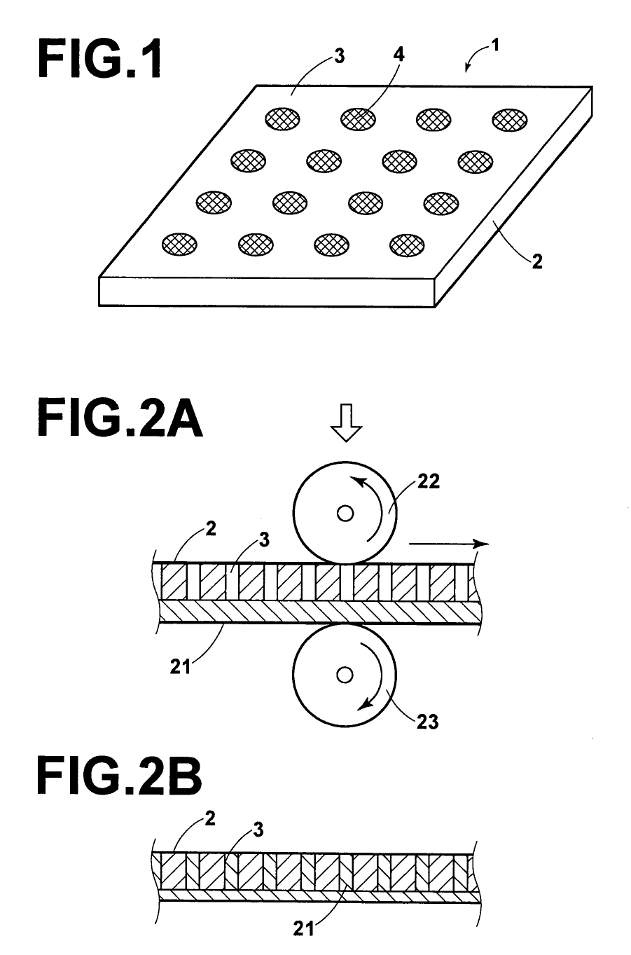

FIG. 1 is a schematic perspective view showing an embodiment of the biochemical analysis unit in accordance with the present invention. With reference to FIG. 1, a biochemical analysis unit 1 comprises a base plate 2, which is provided with a plurality of holes 3, 3, . . . , and a plurality of adsorptive regions 4, 4, . . . , each of which is filled in one of the holes 3, 3, . . . and comprises a porous material adhered to the base plate 2.

Such that light scattering may be prevented from occurring within the biochemical analysis unit 1, the base plate 2 should preferably be made from a material, which does not transmit radiation or light, or which attenuates radiation or light. The material for the formation of the base plate 2 should preferably be a metal or a ceramic material. Also, in cases where a plastic material, for which the hole making processing is capable of being performed easily, is employed as the material for the formation of the base plate 2, particles should preferably be dispersed within the plastic material, such that radiation or light is capable of being attenuated even further.

Examples of the metals, which may be utilized preferably for the formation of the base plate 2, include copper, silver, gold, zinc, lead, aluminum, titanium, tin, chromium, iron, nickel, cobalt, tantalum, and alloys, such as stainless steel and bronze. Examples of the ceramic materials, which may be utilized preferably for the formation of the base plate 2, include alumina, zirconia, magnesia, and quartz. Examples of the plastic materials, which may be utilized preferably for the formation of the base plate 2, include polyolefins, such as a polyethylene and a polypropylene; polystyrenes; acrylic resins, such as a polymethylmethacrylate; polyvinyl chlorides; polyvinylidene chlorides; polyvinylidene fluorides; polytetrafluoroethylenes; polychlorotrifluoroethylenes; polycarbonates; polyesters, such as a polyethylene naphthalate and a polyethylene terephthalate; aliphatic polyamides, such as a 6-nylon and a 6,6-nylon; polyimides; polysulfones; polyphenylene sulfides; silicon resins, such as a polydiphenyl siloxane; phenolic resins, such as novolak; epoxy resins; polyurethanes; celluloses, such as cellulose acetate and nitrocellulose; copolymers, such as a butadiene-styrene copolymer; and blends of plastic materials.

In cases where the base plate 2 is constituted of a plastic material, in order for radiation or light to be attenuated, the plastic material should preferably be loaded with particles of metal oxides, glass fibers, or the like. Examples of the metal oxides include silicon dioxide, alumina, titanium dioxide, iron oxide, and copper oxide. However, the metal oxides are not limited to those enumerated above.

The radiation attenuating properties or the light attenuating properties should preferably be such that, when radiation or light, which is radiated out from the labeled receptor or the labeled ligand having been bound to the ligand or the receptor at the surface or the interior of the porous adsorptive material having been filled in one of the holes 3, 3, . . . of the base plate 2, has passed from the hole 3 through the base plate wall to the adjacent hole 3, the intensity of the radiation or the light reduces to an intensity of at most ⅕ of the original intensity. The radiation attenuating properties or the light attenuating properties should more preferably be such that the intensity of the radiation or the light having passed through the base plate wall in the manner described above reduces to an intensity of at most 1/10 of the original intensity.

In order for the radiation, such as electron rays, coming from a sample having been labeled with the radioactive labeling substance to be blocked efficiently, the mean density of the base plate 2 may ordinarily be at least 0.6 g/cm³. The mean density of the base plate 2 should preferably fall within the range of 1 g/cm³ to 20 g/cm³, and should more preferably fall within the range of 2 g/cm³ to log/cm³. Since the transmission distance of the electron rays is in inverse proportion to the density, in cases where the radioactive labeling substance is an ordinary radioactive isotope (RI), such as ³²P, ³³P, ³⁵S, or ¹⁴C, and the mean density of the base plate 2 falls within the range described above, the electron rays coming from the RI of the sample, which is fixed within each of the holes 3, 3, is capable of being blocked by the partition wall of the base plate 2, and the problems are capable of being prevented from occurring in that resolution of a radiation image is adversely affected by transmission and scattering of the electron rays.

The thickness of the base plate 2 may ordinarily fall within the range of 50 μm to 1, 000 μm, and should preferably fall within the range of 100 μm to 500 μm.

Such that the density of the holes 3, 3, . . . made through the base plate 2 may be enhanced, the area (size) of the opening of each of the holes 3, 3, . . . may ordinarily be smaller than 5. The area of the opening of each of the holes 3, 3, . . . should preferably be smaller than 1 mm², should more preferably be smaller than 0.33 mm², and should most preferably be smaller than 0.01 mm². Also, the area of the opening of each of the holes 3, 3, . . . should preferably be at least 0.001 mm².

The pitch of the holes 3, 3, . . . (i.e., the distance between the center points of two holes which are adjacent to each other) should preferably fall within the range of 0.05 mm to 3 mm. Also, the spacing between two adjacent holes 3, 3 (i.e., the shortest distance between edges of two adjacent holes 3, 3) should preferably fall within the range of 0.01 mm to 1.5 mm. The number (the array density) of the holes 3, 3, . . . may ordinarily be at least 10 holes/cm². The number (the array density) of the holes 3, 3, . . . should preferably be at least 100 holes/cm², should more preferably be at least 500 holes/cm², and should most preferably be at least 1,000 holes/cm². Also, the number (the array density) of the holes 3, 3, . . . should preferably be at most 100,000 holes/cm², and should more preferably be at most 10,000 holes/cm². The holes 3, 3, . . . need not necessarily be arrayed at equal spacing as illustrated in FIG. 1. For example, the holes 3, 3, . . . may be grouped into several number of blocks (units) comprising a plurality of holes and may be formed in units of the blocks.

Perforation of the plurality of the holes 3, 3, . . . through the base plate 2 may be performed with, for example, a punching technique for punching with a pin, a technique for electrical discharge machining, in which a pulsed high voltage is applied across electrodes in order to volatilize the base plate material, an etching technique, or a laser beam irradiation technique. In cases where the material of the base plate is a metal material or a plastic material, the biochemical analysis unit may be prepared with an operation for performing corona discharge or plasma discharge on the surface of the base plate, applying an adhesive agent to the surface of the base plate, and laminating the porous material for the formation of the adsorptive regions by use of means, such as a press. Also, in cases where the porous material for the formation of the adsorptive regions is pressed against the base plate, the base plate and the porous material for the formation of the adsorptive regions may be divided previously into a plurality of sheets, and the plurality of the sheets may be pressed intermittently. Alternatively, a long web of the base plate and a long web of the porous material for the formation of the adsorptive regions may be conveyed continuously between two rolls.

FIGS. 2A and 2B are schematic views showing an example of how the biochemical analysis unit in accordance with the present invention is produced. In the example shown in FIGS. 2A and 2B, the biochemical analysis unit is produced with a pressing technique, wherein a porous film 21 and the base plate 2 are superposed one upon the other and pressed together, and the porous film 21 is thereby press-fitted into the holes 3, 3, . . . of the base plate 2. As the porous film 21, a commercially available porous film having a pore diameter falling within the range of 1 μm to 10 μm may be utilized. The pore diameter of the porous film 21 should preferably fall within the range of 1 μm to 5 μm, and should more preferably fall within the range of 2 μm to 4 μm. With the pressing technique, the porous film 21 is capable of being press-fitted into the holes 3, 3, . . . of the base plate 2 such that little change occurs with the pore diameter of pores of the region of the porous film 21, which region is press-fitted into each of the holes 3, 3, . . .

As illustrated in FIG. 2A, the porous film 21 and the base plate 2 having the holes 3, 3, . . . are superposed one upon the other and pressed together by being passed between a press roll 22 and a back-up roll 23. In this manner, as illustrated in FIG. 2B, the porous film 21 is press-fitted into the holes 3, 3, . . . of the base plate 2. In such cases, the porous film 21 may be softened with a technique wherein, for example, the press roll 22 and the back-up roll 23 are heated.

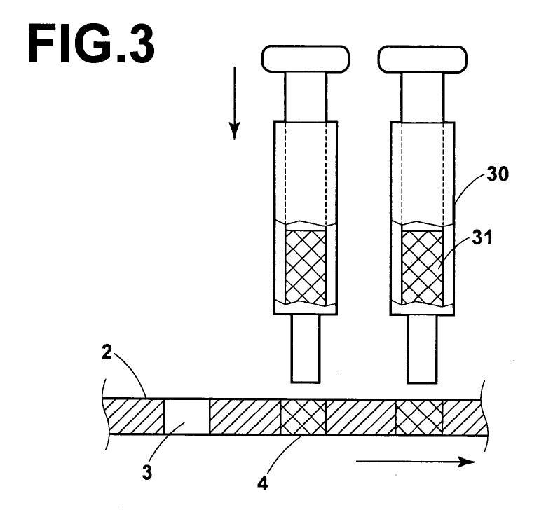

Alternatively, the biochemical analysis unit in accordance with the present invention may be produced with a technique, wherein a solution (hereinbelow referred to as the dope) containing a porous material in a solvent is injected into the holes 3, 3, . . . of the base plate 2. FIG. 3 is a schematic view showing a different example of how the biochemical analysis unit in accordance with the present invention is produced. As illustrated in FIG. 3, a dispenser 30 for injecting a dope 31 into the holes 3, 3, . . . of the base plate 2 is located above the base plate 2, which is conveyed continuously or intermittently. The dispenser 30 intermittently injects the dope 31 into each of the holes 3, 3, . . . of the base plate 2. After the dope 31 has been injected into each of the holes 3, 3, . . . of the base plate 2, air having a controlled temperature and a controlled humidity is fed over the base plate 2 at a predetermined flow rate, and the solvent contained in the dope 31 is vaporized little by little. In this manner, pores are capable of being formed. Alternatively, a porous film may be formed with a different process, wherein the dope is cast or coated on a support, and the resulting casting layer or the resulting coating layer is then dipped in a bad solvent for the polymer of the porous film or in a mixed solvent of a good solvent or a bad solvent for the polymer and thereafter subjected to washing with water and drying. As another alternative, a porous film may be formed with a different process, wherein the dope is castor coated on a support, and the resulting casting layer or the resulting coating layer is then dried little by little.

In the biochemical analysis unit in accordance with the present invention, as the porous material for the formation of the adsorptive regions of the biochemical analysis unit, a porous quality material or a fiber material may be utilized preferably. The porous quality material and the fiber material may be utilized in combination in order to form the adsorptive regions of the biochemical analysis unit. In the biochemical analysis unit in accordance with the present invention, the porous material, which may be utilized for the formation of the adsorptive regions of the biochemical analysis unit, may be an organic material, an inorganic material, or an organic-inorganic composite material.

The organic porous quality material, which may be utilized for the formation of the adsorptive regions of the biochemical analysis unit, may be selected from a wide variety of materials. However, the organic porous quality material should preferably be a carbon porous quality material, such as active carbon, or a porous quality material capable of forming a membrane filter. As the porous quality material capable of forming a membrane filter, a polymer soluble in a solvent should preferably be utilized. Examples of the polymers soluble in a solvent include cellulose derivatives, such as nitrocellulose, regenerated cellulose, cellulose acetate, and cellulose acetate butyrate; aliphatic polyamides, such as a 6-nylon, a 6,6-nylon, and a 4,10-nylon; polyolefins, such as a polyethylene and a polypropylene; chlorine-containing polymers, such as a polyvinyl chloride and a polyvinylidene chloride; fluorine resins, such as a polyvinylidene fluoride and a polytetrafluoride; polycarbonates; polysulfones; alginic acids and alginic acid derivatives, such as alginic acid, calcium alginate, and an alginic acid-polylysine polyion complex; and collagen. Copolymers or composite materials (mixture materials) of the above-enumerated polymers may also be utilized.

The fiber material, which may be utilized for the formation of the adsorptive regions of the biochemical analysis unit, maybe selected from a wide variety of materials. Examples of the fiber materials, which may be utilized preferably, include the cellulose derivatives and the aliphatic polyamides enumerated above.

The inorganic porous quality material, which may be utilized for the formation of the adsorptive regions of the biochemical analysis unit, may be selected from a wide variety of materials. Examples of the inorganic porous quality materials, which may be utilized preferably, include metals, such as platinum, gold, iron, silver, nickel, and aluminum; oxides of metals, and the like, such as alumina, silica, titania, and zeolite; metal salts, such as hydroxyapatite and calcium sulfate; and composite materials of the above-enumerated materials.

In cases where a commercially available porous film is utilized as the porous film, a porous film conforming to a pore diameter specification of 1 μm to 10 μm may be utilized. A porous film conforming to a pore diameter specification of 1 μm to 5 μm should preferably be utilized, and a porous film conforming to a pore diameter specification of 2 μm to 4 μm should more preferably be utilized. Specifically, for example, Biodyne A (pore diameter: 1.2 μm), Biodyne A (pore diameter: 3 μm), or Biodyne A (pore diameter: 5 μm), which is supplied by Paul Co., Ltd., may be utilized as the porous film. In cases where the adsorptive regions have been formed with, for example, the technique described above, wherein the porous quality material is injected into the holes, the pore diameter of the adsorptive regions is capable of being measured with the technique described below, and the pore diameter is capable of being adjusted by appropriate alteration of conditions for the formation of the adsorptive regions.

The pore diameter of the adsorptive regions is capable of being measured with a porous material automatic pore measuring system supplied by Porous Materials Inc. (PMI). The pore diameter of the adsorptive regions may be measured in the manner described below. Specifically, a porous adsorptive region is wetted with a test liquid. Also, air is fed to the wetted porous adsorptive region, and the air pressure is raised little by little. In this manner, an air permeation flow rate is measured. (A wet flow rate curve is thus formed.) Further, in the state in which the adsorptive region is not wetted with the test liquid, the air permeation flow rate is measured at the same pressure. (A dry flow rate curve is thus formed.) The wet flow rate and the dry flow rate are then compared with each other. (Specifically, the pressure associated with a point, at which a curve having an inclination of one half of the inclination of the dry flow rate curve and the wet flow rate curve intersect with each other, is found.) A mean pore diameter is then capable of being calculated from a relation between the pressure and the pore diameter.

How a biochemical analysis using the biochemical analysis unit in accordance with the present invention is performed will be described hereinbelow by taking a chemical luminescence technique as an example.

In the chemical luminescence technique using the biochemical analysis unit in accordance with the present invention, firstly, the ligands or the receptors are bound respectively to the adsorptive regions of the biochemical analysis unit, which is provided with the plurality of the porous adsorptive regions.

Examples of the ligands or the receptors, which are bound respectively to the porous adsorptive regions of the biochemical analysis unit, include hormones, tumor markers, enzymes, antibodies, antigens, abzymes, other proteins, nucleic acids, cDNA's, DNA's, and RNA's, whose characteristics, compositions, structures, base sequences, base lengths, and the like, are known. After the ligands or the receptors have been spotted respectively onto the adsorptive regions of the biochemical analysis unit, the ligands or the receptors are capable of being fixed to the adsorptive regions with ultraviolet light irradiation, or the like. In cases where the aforesaid biochemical analysis unit, in which the ligands or the receptors have already been bound respectively to the porous adsorptive regions, is utilized, the steps of spotting and fixing the ligands or the receptors are omitted.

Thereafter, a labeled receptor or a labeled ligand, which has been labeled with a labeling substance, is subjected to specific binding with the ligands or the receptors, each of which has been bound to one of the porous adsorptive regions of the biochemical analysis unit. The labeled receptor or the labeled ligand is thus specifically bound to at least one of the ligands or at least one of the receptors. The labeled receptor or the labeled ligand is the substance, which has been sampled from an organism through extraction, isolation, or the like, or has been subjected to chemical treatment after being sampled, and which has been labeled with the labeling substance. The labeled receptor or the labeled ligand is capable of undergoing the specific binding with at least one of the ligands, each of which has been bound to one of the porous adsorptive regions of the biochemical analysis unit, or at least one of the receptors, each of which has been bound to one of the porous adsorptive regions of the biochemical analysis unit. Examples of the labeled receptors or the labeled ligands include hormones, tumor markers, enzymes, antibodies, antigens, abzymes, other proteins, nucleic acids, DNA's, and mRNA's. Examples of preferable labeling substances, with which the receptors or the ligands may be labeled, include antigens, such as digoxigenin, biotin, avidin, and fluorescein, and antibodies with respect to the above-enumerated antigens.

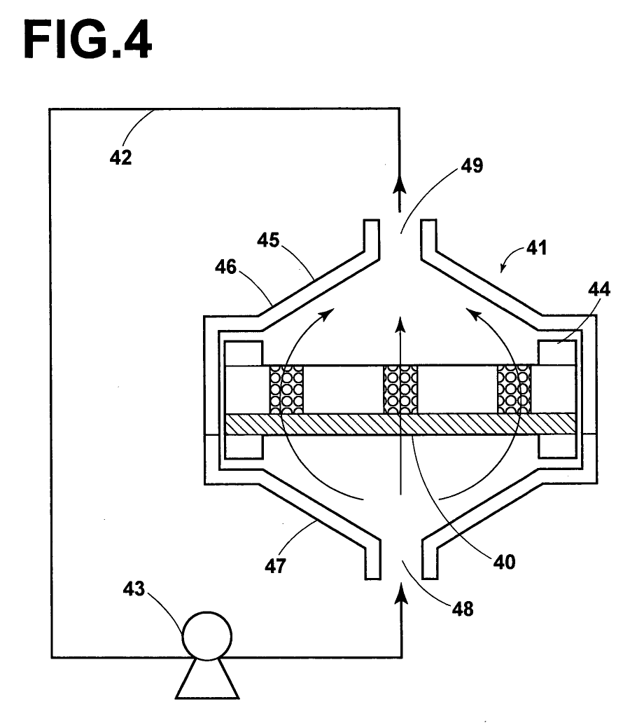

After the labeled receptor or the labeled ligand has been specifically bound to at least one of the ligands, each of which has been bound to one of the porous adsorptive regions of the biochemical analysis unit, or at least one of the receptors, each of which has been bound to one of the porous adsorptive regions of the biochemical analysis unit, the biochemical analysis unit is set within, for example, the reaction vessel, which is illustrated in FIG. 4 and in which the reaction liquid is capable of being forcibly caused to flow such that the reaction liquid flows across each of the adsorptive regions of the biochemical analysis unit.

FIG. 4 is a schematic sectional view showing an example of a reactor, which is employed for the chemical luminescence technique using the biochemical analysis unit in accordance with the present invention, and in which a reaction liquid is forcibly caused to flow. With reference to FIG. 4, the reactor comprises a reaction vessel 41, a reaction liquid circulating pipe 42 and a pump 43. The reaction vessel 41 is provided with a biochemical analysis unit support section 44, which supports a biochemical analysis unit 40 and has sealing functions for preventing liquid leakage. A reaction vessel main body 45 of the reaction vessel 41 comprises a reaction vessel upper half 46 and a reaction vessel lower half 47. The reaction vessel upper half 46 is releasably secured to the reaction vessel main body 45. When the biochemical analysis unit 40 is to be set within the reaction vessel 41, the reaction vessel upper half 46 is dismounted from the reaction vessel main body 45, and the biochemical analysis unit 40 is set within the reaction vessel 41. A bottom wall of the reaction vessel lower half 47 is provided with a reaction liquid inlet 48, through which a reaction liquid is capable of flowing. Also, a top wall of the reaction vessel upper half 46 is provided with a reaction liquid outlet 49, through which the reaction liquid is capable of flowing. Further, the reaction liquid circulating pipe 42 is releasably fitted to the reaction liquid inlet 48 and the reaction liquid outlet 49 of the reaction vessel 41. The reactor is constituted such that the reaction liquid is introduced by the pump 43 into the reaction vessel main body 45 through the reaction liquid inlet 48, passed through the biochemical analysis unit 40, discharged through the reaction liquid outlet 49, and circulated through the reaction liquid circulating pipe 42.

In this example, the biochemical analysis unit in accordance with the present invention is set in the reaction vessel, which is capable of forcibly causing the reaction liquid to flow such that the reaction liquid flows across each of the adsorptive regions of the biochemical analysis unit. However, the biochemical analysis unit in accordance with the present invention is not limited to the use within the reaction vessel described above. For example, the biochemical analysis unit in accordance with the present invention may be utilized for the shaking technique, wherein the biochemical analysis unit and the reaction liquid are put into a hybridization bag, vibrations are given to the hybridization bag, and the labeled receptor or the labeled ligand is thus moved through convection or diffusion and is specifically bound to one of the ligands or the receptors having been fixed to the adsorptive regions of the biochemical analysis unit.

In order for the labeled receptor or the labeled ligand, which has not been specifically bound to the ligands or the receptors having been bound respectively to the porous adsorptive regions of the biochemical analysis unit, to be removed, the biochemical analysis unit having been set within the reaction vessel should preferably be washed with a technique for forcibly causing a washing liquid to flow across each of the adsorptive regions. In such cases, since the washing liquid is forcibly caused to flow across each of the adsorptive regions, the labeled receptor or the labeled ligand, which has not been specifically bound to the ligands or the receptors having been bound respectively to the porous adsorptive regions of the biochemical analysis unit, is capable of being peeled off and removed efficiently. Therefore, the washing efficiency is capable of being enhanced markedly.

After the reaction liquid, which contains the enzyme-labeled antibody, is forcibly caused to flow such that the reaction liquid flows across each of the adsorptive regions of the biochemical analysis unit, and the enzyme-labeled antibody is thus subjected to the specific binding with the labeled receptor or the labeled ligand, the enzyme-labeled antibody, which has not been specifically bound to the labeled receptor or the labeled ligand, may be removed. In cases where the enzyme-labeled antibody, which has not been specifically bound to the labeled receptor or the labeled ligand, is to be removed, the washing process described above should preferably be performed. In this manner, the enzyme-labeled antibody, which has not been specifically bound to the labeled receptor or the labeled ligand, is capable of being peeled off and removed efficiently. Therefore, the washing efficiency is capable of being enhanced markedly.

Before the enzyme-labeled antibody is subjected to the specific binding with the labeled receptor or the labeled ligand having been specifically bound to at least one of the ligands, each of which has been bound to one of the porous adsorptive regions of the biochemical analysis unit, or at least one of the receptors, each of which has been bound to one of the porous adsorptive regions of the biochemical analysis unit, the adsorptive regions should preferably be blocked with a blocking process, wherein a blocking buffer with respect to the enzyme-labeled antibody is forcibly caused to flow such that the blocking buffer flows across each of the adsorptive regions. With the blocking process, the problems are capable of being prevented from occurring in that, instead of the enzyme-labeled antibody being subjected to the specific binding with the antigen of the labeled receptor or the labeled ligand, the enzyme-labeled antibody is directly bound to the adsorptive regions of the biochemical analysis unit.

Thereafter, the reaction liquid, which contains the enzyme-labeled antibody, is forcibly caused to flow such that the reaction liquid flows across each of the adsorptive regions of the biochemical analysis unit, and the enzyme-labeled antibody is thus subjected to the specific binding with the labeled receptor or the labeled ligand. The enzyme-labeled antibody is the antibody with respect to the labeling substance of the labeled receptor or the labeled ligand, which antibody has been labeled with an enzyme. (In cases where the labeling substance of the labeled receptor or the labeled ligand is an antibody, the enzyme-labeled antibody is the antigen with respect to the labeling substance of the labeled receptor or the labeled ligand, which antigen has been labeled with an enzyme.) Examples of the enzymes preferable as the enzyme of the enzyme-labeled antibody include alkaline phosphatase, peroxidase, and luciferase.

Thereafter, the biochemical analysis unit is taken out from the reaction vessel, and a chemical luminescence substrate is brought into contact with the enzyme-labeled antibody, which has been specifically bound to the labeled receptor or the labeled ligand. In cases where the enzyme of the enzyme-labeled antibody is alkaline phosphatase, the chemical luminescence substrate caused to undergo the reaction with the enzyme-labeled antibody may be dioxetane. In cases where the enzyme of the enzyme-labeled antibody is peroxidase, the chemical luminescence substrate caused to undergo the reaction with the enzyme-labeled antibody maybe luminol. Also, in where the enzyme of the enzyme-labeled antibody is luciferase, the chemical luminescence substrate caused to undergo the reaction with the enzyme-labeled antibody may be luciferin. However, the chemical luminescence substrate caused to undergo the reaction with the enzyme-labeled antibody is not limited to the chemical luminescence substrates enumerated above.

In cases where the chemical luminescence substrate and the enzyme are brought into contact with each other, the chemical luminescence having wavelengths falling within the visible light wavelength range is produced. Therefore, the produced chemical luminescence may be detected photoelectrically, and the image data for a biochemical analysis may be formed in accordance with the detected chemical luminescence. In this manner, the labeled receptor or the labeled ligand is capable of being detected and determined.

The present invention will further be illustrated by the following nonlimitative examples.

### EXAMPLES

### Example 1

With an etching technique, 6,400 fine holes were formed in a SUS304 sheet (acting as a base plate material sheet) having a size of 80 mm×80 mm and a thickness of 100 μm. Each of the fine holes had a circular opening region having a hole diameter of 0.2 mm. The fine holes were formed at a hole pitch of 0.3 mm and a hole spacing of 0.1 mm. The fine holes were formed with 10×10 holes being taken as one unit.

Thereafter, an adhesive agent was applied to one surface of the base plate material sheet, and the adhesive agent, which entered into the holes having been formed in the base plate material sheet, was removed by suction. The adhesive agent remaining on the surface of the base plate material sheet was then dried. Thereafter, Biodyne A (pore diameter: 1.2 μm, supplied by Paul Co., Ltd.) was superposed upon the surface of the base plate material sheet, which surface had been coated with the adhesive agent. The combination of Biodyne A and the base plate material sheet was then heated to a temperature of 150° C. and pressed under pressure such that the pressure per 1 cm² was 300 kg. Biodyne A was thus press-fitted into the fine holes of the base plate material sheet. In this manner, a biochemical analysis unit, which comprised a stainless steel barrier wall and the plurality of polymer-filled regions formed in the fine holes, was prepared.

Also, after a molecular weight marker pBR328/BgII, HinfI (250 nl/μg, supplied by Roche Diagnostics K.K.) having been dissolved in the TE buffer was boiled for five minutes, the liquid was cooled for one minute in an ice-bath, and the pBR328/BgII, HinfI was thus converted into a single stranded form. The thus obtained pBR328/BgII, HinfI liquid was then spotted onto the adsorptive regions of the biochemical analysis unit having been prepared in the manner described above. Thereafter, with irradiation of ultraviolet light (254 nm, 33 mJ/cm²), the single stranded pBR328/BgII, HinfI was fixed to the adsorptive regions of the biochemical analysis unit.

Thereafter, 10 pg of a digoxigenin-labeled (DIG-labeled) pBR328-DNA liquid (supplied by Roche Diagnostics K.K.) was subjected to thermal denaturation and added to 5 ml of a hybridization buffer (6×SSC, 0.01 M EDTA, 5× denhardt's solution, 0.5% SDS, 100 μg Sheared, denatured salmon sperm DNA).

The biochemical analysis unit described above was secured to the reactor illustrated in FIG. 4, which was capable of forcibly causing a reaction liquid to flow. Also, 5 ml of a pre-hybridization buffer (the same buffer as the hybridization buffer described above) at a temperature of 65° C. was circulated within the reactor for one hour (linear speed: 0.2 cm/sec). Thereafter, the hybridization buffer, to which the DIG-labeled pBR328-DNA liquid had been added, was circulated within the reactor at a temperature of 65° C. for 18 hours, and hybridization was thus performed. A circulation washing operation was performed, wherein two washing steps were performed for five minutes per washing step by use of washing buffer 1 (2×SSC, 0.1% SDS), and wherein two washing steps were performed for five minutes per washing step by use of washing buffer 2 (0.1×SSC, 0.1% SDS). (During the circulation washing operation, the buffer temperature was 65° C.)

A blocking buffer (DIG, described in “Wash and Block buffer Set” and supplied by Roche Diagnostics K.K.) was subjected to filtration using an Ultrafree filter having a pore diameter of 0.22 μm (supplied by Millipore Co., Ltd.). The blocking buffer after being subjected to the filtration was circulated within the reactor at room temperature for 10 minutes, and thereafter the circulation was ceased for 50 minutes. Thereafter, an alkaline phosphatase-labeled DIG antibody, which had been subjected to filtration using the Ultrafree filter having a pore diameter of 0.22 μm (supplied by Millipore Co., Ltd.), was diluted with the blocking buffer, which had been subjected to filtration using the Ultrafree filter having a pore diameter of 0.22 μm. The resulting dilute liquid was circulated within the reactor at room temperature for one minute, and thereafter the circulation was ceased for 60 minutes.

Thereafter, a chemiluminescent washing liquid (DIG, described in “Wash and Block buffer Set” and supplied by Roche Diagnostics K.K.) was circulated within the reactor at room temperature for 15 minutes. The operation for circulating the chemiluminescent washing liquid within the reactor was iterated three times. Thereafter, the biochemical analysis unit was dipped in a detection buffer (DIG, described in “Wash and Block buffer Set” and supplied by Roche Diagnostics K.K.) for five minutes and was then brought into contact with a liquid containing a chemical luminescence substrate (CDP-star, ready to use, supplied by Roche Diagnostics K.K.) for one hour. Also, the chemical luminescence, which was emitted from the adsorptive regions of the biochemical analysis unit, was detected photoelectrically by use of a cooled CCD camera (LAS1000, supplied by Fuji Photo Film Co., Ltd.).

### Example 2

A chemiluminescence operation was performed in the same manner as that in Example 1, except that Biodyne A (pore diameter: 3 μm, supplied by Paul Co., Ltd.) was utilized for the formation of a biochemical analysis unit. Also, the chemical luminescence, which was emitted from the adsorptive regions of the biochemical analysis unit, was detected in the same manner as that in Example 1.

### Example 3

A chemiluminescence operation was performed in the same manner as that in Example 1, except that Biodyne A (pore diameter: 5 μm, supplied by Paul Co., Ltd.) was utilized for the formation of a biochemical analysis unit. Also, the chemical luminescence, which was emitted from the adsorptive regions of the biochemical analysis unit, was detected in the same manner as that in Example 1.

### Comparative Example 1

A chemiluminescence operation was performed in the same manner as that in Example 1, except that Biodyne A (pore diameter: 0.45 μm, supplied by Paul Co., Ltd.) was utilized for the formation of a biochemical analysis unit. Also, the chemical luminescence, which was emitted from the adsorptive regions of the biochemical analysis unit, was detected in the same manner as that in Example 1.

With each of the biochemical analysis units formed in Examples 1, 2, 3 and Comparative Example 1, the intensity of the signal, the intensity of the background, and the signal-to-noise ratio (S/N ratio) listed in Table 1 below were obtained.

TABLE 1

| | Signal | Background | S/N |
|---|---|---|---|
| Ex. 1 | 1,888,000 | 8,740 | 216 |
| Ex. 2 | 2,404,000 | 8,320 | 289 |
| Ex. 3 | 2,055,000 | 9,430 | 218 |
| Comp. | 1,434,000 | 14,900 | 96 |
| Ex. 1 | | | |

As clear from Table 1, with each of the biochemical analysis units formed in Examples 1, 2, and 3, the intensity of the signal is higher than the intensity of the signal obtained with the biochemical analysis unit formed in Comparative Example 1, and the intensity of the background was lower than the intensity of the background obtained with the biochemical analysis unit formed in Comparative Example 1. With each of the biochemical analysis units formed in Examples 1, 2, and 3, wherein the pore diameter of the porous adsorptive material falls within the range of 1 μm to 5 μm, the problems are capable of being prevented from occurring in that the labeled receptor or the labeled ligand, or the like, is bound to the adsorptive regions in a non-specific manner. Also, the surface area of each of the adsorptive regions is capable of being adjusted appropriately. Therefore, the problems are capable of being prevented from occurring in that the amount of each of the ligands or the receptors capable of being fixed to one of the adsorptive regions becomes small.

In the embodiments and Examples described above, the biochemical analysis unit in accordance with the present invention is utilized for the chemical luminescence technique. However, the biochemical analysis unit in accordance with the present invention is capable of being utilized also in cases where a substance having been labeled with a radio active labeling substance or a fluorescent labeling substance is to be detected. Also, besides the circulation technique, wherein the reaction liquid is forcibly caused to circulate such that the reaction liquid flows across each of the adsorptive regions of the biochemical analysis unit, the biochemical analysis unit in accordance with the present invention is capable of being utilized also for the hybridization, or the like, performed with the conventional shaking technique. In such cases, the intensity of the background is capable of being suppressed, and noise is thus capable of being suppressed, while the detection limit is being kept high.

What is claimed is:

1. A biochemical analysis unit, comprising:
i) a base plate, which has a plurality of holes and is constituted of a material having radiation attenuating properties and/or light attenuating properties, and
ii) a porous adsorptive material, which is filled in each of the plurality of the holes of the base plate and forms each of a plurality of adsorptive regions,
wherein the porous adsorptive material, which forms each of the plurality of the adsorptive regions, has a pore diameter falling within the range of 1 μm to 10 μm.

2. A biochemical analysis unit as defined in claim 1 wherein the porous adsorptive material takes on the form of a film.

3. A biochemical analysis unit as defined in claim 1 wherein the porous adsorptive material, which forms each of the plurality of the adsorptive regions, has a pore diameter falling within the range of 1 μm to 5 μm.

4. A biochemical analysis unit as defined in claim 2 wherein the porous adsorptive material, which forms each of the plurality of the adsorptive regions, has a pore diameter falling within the range of 1 μm to 5 μm.

5. A biochemical analysis unit as defined in claim 3 wherein the porous adsorptive material, which forms each of the plurality of the adsorptive regions, has a pore diameter falling within the range of 2 μm to 4 μm.

6. A biochemical analysis unit as defined in claim 4 wherein the porous adsorptive material, which forms each of the plurality of the adsorptive regions, has a pore diameter falling within the range of 2 μm to 4 μm.
